(19) United States
(12) Patent Application Publication
(10) Pub. No.: US 2023/0034756 A1
(43) Pub. Date: Feb. 2, 2023

(54) CELL ANALYSIS METHOD

(71) Applicant: HAMAMATSU PHOTONICS K.K., Hamamatsu-shi, Shizuoka (JP)

(72) Inventors: Kazuhiko FUJIWARA, Hamamatsu-shi, Shizuoka (JP); Sayaka KAZAMI, Hamamatsu-shi, Shizuoka (JP); Yoshihiro MARUYAMA, Hamamatsu-shi, Shizuoka (JP)

(73) Assignee: HAMAMATSU PHOTONICS K.K., Hamamatsu-shi, Shizuoka (JP)

(21) Appl. No.: 17/791,280

(22) PCT Filed: Jan. 14, 2021
(86) PCT No.: PCT/JP2021/001046

§ 371 Date: Jul. 7, 2022

(30) Foreign Application Priority Data
Jan. 27, 2020 (JP) 2020-010708

(51) Int. Cl. G01N 33/569; G01N 21/65

(52) U.S. Cl. CPC G01N 33/569; G01N 21/658

(57) ABSTRACT

A cell analysis method includes a mixing step of mixing a cell as an analyte, a metal ion solution, and a reducing agent to prepare a mixture solution, a metal microstructure generation step of reducing metal ions in the mixture solution by reducing action of the reducing agent to generate a metal microstructure on a support, and attaching the cell or a cell-derived substance to the metal microstructure, a drying step of, after the metal microstructure generation step, drying the support, a measurement step of, after the drying step, irradiating the metal microstructure on the support with excitation light, and measuring a spectrum of Raman scattered light generated by the excitation light irradiation, and an analysis step of analyzing the cell based on the spectrum of the Raman scattered light.

### TECHNICAL FIELD

The present disclosure relates to a cell analysis method.

### BACKGROUND ART

As a method for analyzing an analyte, a method based on a spectrum of Raman scattered light generated by irradiating the analyte with excitation light is known. Since the Raman scattering spectrum reflects molecular vibrations of the analyte, it is possible to analyze the analyte based on a shape of the Raman scattering spectrum. However, in this analysis method, a Raman scattering efficiency is very small in general, and therefore, it is difficult to perform the analysis when an amount of analytes is very small. Accordingly, conventionally, the types of analytes that can be practically subjected to this analysis method have been limited to substances such as minerals and high density plastics.

Meanwhile, surface enhanced Raman scattering (SERS) spectroscopy has a significantly improved Raman scattering efficiency, and is capable of high sensitivity measurement, and thus, it is expected to be capable of analyzing a low concentration sample and it attracts attention. In the SERS spectroscopy, high intensity Raman scattered light can be generated from the analyte, in a case where two principal conditions are satisfied, that is, an enhanced electric field (photon field) is generated at a metal microstructure irradiated with excitation light (first condition), and the analyte constantly exists in the immediate vicinity of the metal microstructure at which the enhanced electric field reaches (second condition).

In order to efficiently satisfy the first condition, a technique including use of a metal microstructure array designed to have various shapes of nanometer-order size has been proposed, in this method, an analyte is analyzed by the SERS spectroscopy by using a substrate (SERS substrate) having a surface provided with the metal microstructure array, and for example, dropping the analyte onto the SERS substrate. Further, there has been proposed another technique of using a dispersion liquid containing metal colloids (for example, silver colloid particles, gold colloid particles) dispersed therein, in this method, an analyte is analyzed by the SERS spectroscopy by putting the analyte into the metal colloid dispersion liquid.

It is necessary to satisfy the above second condition to analyze the analyte by the SERS spectroscopy, in the case of using the SERS substrate and also in the case of using the metal colloid dispersion liquid. That is, the enhanced electric field can be achieved in a spatially limited area depending on the metal microstructure, and in many cases, such area exists in a gap in the metal microstructure. Therefore, in order to efficiently generate SERS light by satisfying the second condition, the analyte needs to exist in the limited gap.

In order to satisfy the second condition, the analyte is required to have high affinity for the metal constituting the metal microstructure and to be easily adsorbed. However, even when the first condition is satisfied by using the SERS substrate with which the enhanced electric field can be efficiently generated, the analyte that has low affinity for the metal constituting the metal microstructure and is difficult to be adsorbed cannot enter the narrow gap in the metal microstructure, and the second condition cannot be satisfied, and thus, it is difficult to analyze the analyte by the SERS spectroscopy.

In order to analyze the analyte by the SERS spectroscopy with use of the SERS substrate or the metal colloid dispersion liquid, it is necessary to prepare the SERS substrate or the metal colloid dispersion liquid in advance. The SERS light is efficiently generated particularly with silver (Ag), however, silver is easily oxidized. When an oxide film is formed on a surface of a silver microstructure on the SERS substrate or silver colloids at the time of spectroscopic measurement, it is not possible to efficiently analyze the analyte by the SERS spectroscopy. Further, it is necessary to keep the SERS substrate or the metal colloids uncontaminated until the spectroscopic measurement starts, and thus, it is not easy to handle these.

Patent Document 1 discloses an invention designed to solve the above-described problem of the conventional techniques. According to the invention disclosed in this document, it is possible to easily perform an analysis by highly efficient SERS spectroscopy.

Further, in Non Patent Document 1, it is described that a Raman scattering spectrum derived from bacteria was obtained by attaching bacteria as an analyte to metal colloid particles and performing the SERS spectroscopy.

### CITATION LIST

### Patent Literature

Patent Document 1: Japanese Patent Application Laid-Open Publication No. 2018-25431

### Non Patent Literature

Non Patent Document 1: Pamela A. Mosier-Boss, “Review on SERS of Bacteria”, Biosensors 2017, 7, 51

### SUMMARY OF INVENTION

### Technical Problem

The invention disclosed in Patent Document 1 can easily perform an analysis of an analyte by highly efficient SERS spectroscopy, however, types of analytes that can be analyzed are limited, and a cell including a bacterium or the like cannot be analyzed as the analyte.

In the technique described in Non Patent Document 1, a metal colloid dispersion liquid is used, and therefore, an analysis of an analyte (a cell including a bacterium or the like) by SERS spectroscopy cannot be performed efficiently and easily.

An object of the present invention is to provide a method capable of easily performing an analysis of a cell being an analyte by highly efficient SERS spectroscopy.

### Solution to Problem

An embodiment of the present invention is a cell analysis method. The cell analysis method includes (1) a mixing step of mixing a cell as an analyte, a metal ion solution, and a reducing agent to prepare a mixture solution; (2) a metal microstructure generation step of reducing metal ions in the mixture solution by reducing action of the reducing agent in the mixture solution to generate a metal microstructure on a support, and attaching the cell or a cell-derived substance to the metal microstructure; (3) a drying step of, after the metal microstructure generation step, drying the support; and (4) a measurement step of, after the drying step, irradiating the metal microstructure on the support with excitation light, and measuring a spectrum of Raman scattered light generated by the excitation light irradiation. Further, the method may further include, between the drying step and the measurement step, a washing step of washing the support.

### Advantageous Effects of Invention

According to the embodiments of the present invention, it is possible to easily perform an analysis of a cell being an analyte by highly efficient SERS spectroscopy.

### BRIEF DESCRIPTION OF DRAWINGS

FIG. 1 is a flowchart of a cell analysis method according to a first embodiment.

FIG. 2 is a flowchart of a cell analysis method according to a second embodiment.

FIG. 3 is a diagram illustrating an optical system of a microspectroscope 1 used for measuring a SERS light spectrum in a measurement step in each example.

FIG. 4 is a table showing samples used in the examples.

FIG. 5 is a diagram showing a SERS light spectrum obtained in the example 1.

FIG. 6 is a diagram showing a SERS light spectrum obtained in the example 2.

FIG. 7 is a diagram showing a SERS light spectrum obtained in the example 3.

FIG. 8 is a diagram showing a SERS light spectrum obtained in the example 4.

FIG. 9 is a diagram showing a SERS light spectrum obtained in the example 5.

FIG. 10 is a photograph of a bright field image in a comparative example.

FIG. 11 is a photograph of a bright field image in the measurement step in the example 2.

FIG. 12 is a photograph of a bright field image in the measurement step in the example 3.

FIG. 13 is a photograph of a bright field image in the measurement step in the example 4.

### DESCRIPTION OF EMBODIMENTS

Hereinafter, embodiments of a cell analysis method will be described in detail with reference to the accompanying drawings. In the description of the drawings, the same elements will be denoted by the same reference signs, and redundant description will be omitted. The present invention is not limited to these examples.

A cell analysis method according to an embodiment prepares a mixture solution by mixing a metal ion solution and a reducing agent, reduces metal ions in the mixture solution by reducing action of the reducing agent in the mixture solution to generate a metal microstructure on a support, and attaches a cell or a cell-derived substance to the metal microstructure. Then, the method irradiates the metal microstructure on the support with excitation light, measures a spectrum of Raman scattered light generated by the excitation light irradiation, and analyzes the cell based on the spectrum of the Raman scattered light. Hereinafter, cell analysis methods according to first and second embodiments will be described.

The cells being the analytes include prokaryotic cells and eukaryotic cells. The prokaryotic cells include bacteria and archaea.

The eukaryotic cells include protists, plants, animals, and fungi. The cell may be a single-cell, a multi-cell, or a cultured cell. The cell-derived substance is generated by cell degradation, and is, for example, contents such as nucleic acid and nucleic acid base contained in the cell or metabolites thereof.

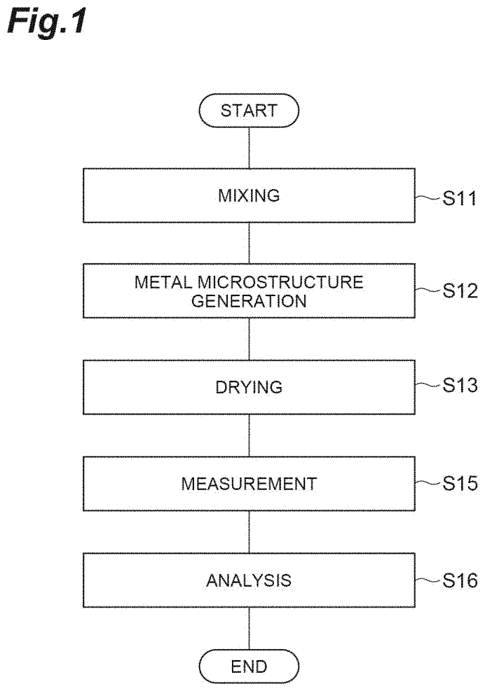

FIG. 1 is a flowchart of a cell analysis method according to a first embodiment. In the cell analysis method of the first embodiment, a mixing step S11, a metal microstructure generation step S12, a drying step S13, a measurement step S15, and an analysis step S16 are sequentially performed to analyze a cell.

In the mixing step S11, a measurement solution containing the cell, a metal ion solution, and a reducing agent are sufficiently mixed to prepare a mixture solution. In addition, a pH adjusting agent may be further mixed to prepare the mixture solution.

The measurement solution, the metal ion solution, the reducing agent, and the pH adjusting agent can be mixed in various ways or in various orders. The measurement solution, the metal ion solution, the reducing agent, and the pH adjusting agent may be mixed at the same time. Further, the measurement solution, the metal ion solution, and the reducing agent may be mixed to prepare an intermediate mixture solution, and then, the intermediate mixture solution and the pH adjusting agent may be mixed to prepare a final mixture solution. Further, a salt may be further mixed. After addition of the pH adjusting agent, the measurement solution may be added thereto even before the metal microstructure is completely generated.

The measurement solution containing the cells is obtained by, for example, dispersing the cells collected by centrifugation after culturing in a liquid culture medium into water (preferably, pure water). The metal ion is arbitrary as long as it can be reduced by the reducing action of the reducing agent, and is, for example, a gold ion or a silver ion. The reducing agent may be, for example, an aqueous glucose solution, an aqueous iron(II) sulfate solution, an aqueous sodium borohydride solution, or an aqueous formaldehyde solution.

The pH adjusting agent is mixed to make the mixture solution alkaline, and is, for example, an aqueous potassium hydroxide solution. The salt is mixed to promote aggregation of metal microparticles, and is, for example, sodium chloride. The amounts and concentrations of the metal ion solution, the reducing agent, and the pH adjusting agent mixed as the final mixture solution are appropriately adjusted according to the amount of the measurement solution and the concentration of the cells in the measurement solution.

In the metal microstructure generation step S12, metal ions in the mixture solution are reduced by the reducing action of the reducing agent in the mixture solution to generate a metal microstructure on a support, and the cell or a cell-derived substance is attached to the metal microstructure. The metal microstructure on the support is a structure in which aggregations of deposited metal microparticles are distributed on the support in the form of islands. In this step, for preventing evaporation of the mixture solution, the support is preferably allowed to stand still for a predetermined time in a humidified environment.

The support may be a container used in preparation of the intermediate mixture solution or the mixture solution, and further, the support may be a substrate prepared separately from the container, and the substrate may be, for example, a glass slide. Further, a glass slide subjected to a water repellent treatment with a predetermined pattern may be used, and the mixture solution may be prepared in an area on the glass slide which is not subjected to the water repellent treatment to generate the metal microstructure. When the substrate prepared separately from the container is used as the support, appropriate amounts of the intermediate mixture solution and the pH adjusting agent are dropped onto the substrate, and the intermediate mixture solution and the pH adjusting agent are sufficiently mixed on the substrate using a micropipette or the like to prepare a final mixture solution, thereby generating the metal microstructure on the substrate.

In the drying step S13, the support on which the metal microstructure is generated is dried. By this drying, the metal microstructure to which the cell or the cell-derived substance is attached aggregates in a limited area on the support.

In the measurement step S15, the metal microstructure on the support is irradiated with excitation light, and a spectrum of Raman scattered light generated by the excitation light irradiation is measured. A measurement direction of the Raman scattered light with respect to an irradiation direction of the excitation light may be arbitrarily selected, any one of backward scattered light and forward scattered light may be measured, and scattered light in any other direction may be measured. Further, an optical filter designed to selectively transmit Raman scattered light is preferably provided in the middle of the measurement optical system.

The excitation light is preferably laser light. An enhanced electric field is generated at the metal microstructure irradiated with the excitation light (first condition), and the cell or the cell-derived substance is attached to the metal microstructure reached by the enhanced electric field (second condition). Thus, the measured Raman scattered light is SERS light generated from the cell or the cell-derived substance.

When the metal microstructure is generated in a narrow area on the support, it is preferable to perform the excitation light irradiation and measure the SERS light spectrum using a microspectroscope. The

SERS light spectrum is measured with the excitation light irradiation in a state where the area on the support in which the metal microstructure is generated is dry.

In the analysis step S16, the cell is analyzed based on the spectrum of the Raman scattered light (SERS light). Specifically, the cell is analyzed based on the position of the Raman shift amount at which a peak appears and the height of the peak in the obtained SERS light spectrum.

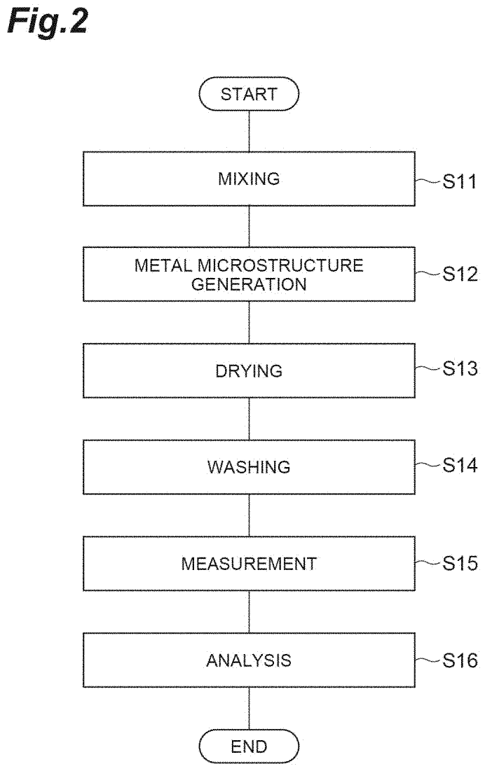

FIG. 2 is a flowchart of a cell analysis method according to a second embodiment. In the cell analysis method of the second embodiment, the mixing step S11, the metal microstructure generation step S12, the drying step S13, a washing step S14, the measurement step S15, and the analysis step S16 are sequentially performed to analyze the cell.

As compared with the cell analysis method of the first embodiment, the cell analysis method of the second embodiment is different in that the washing step S14 is performed between the drying step S13 and the measurement step S15. In the washing step S14, the support dried in the drying step S13 is washed with water (preferably, pure water) to remove the salt remaining in the reaction mixture, and then the support is dried again. By this drying, the metal microstructure to which the cell or the cell-derived substance is attached aggregates in a limited area on the support.

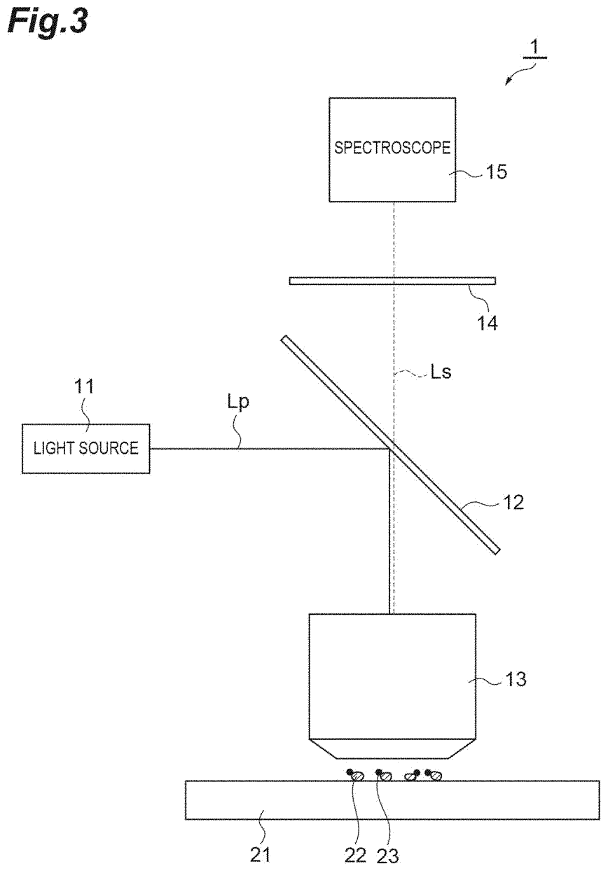

Next, examples 1 to 5 will be described. FIG. 3 is a diagram illustrating an optical system of a microspectroscope 1 used for measuring the SERS light spectrum in the measurement step in each example. In each example, a glass slide was used as the support for supporting the metal microstructure. On a surface of the support (glass slide) 21, a metal microstructure 22 in which aggregations of deposited metal microparticles were distributed in the form of islands was formed. A cell (or a cell-derived substance) 23 was attached to the metal microstructure 22.

A semiconductor laser light source which outputs laser light having a wavelength of 640 nm as excitation light Lp was used as an excitation light source 11. The excitation light Lp output from the excitation light source 11 was reflected by a dichroic mirror 12, and then wan transmitted through an objective lens 13 to irradiate the metal microstructure 22 and the cell 23. As the objective lens 13, one having a magnification of 100× and a numerical aperture of 0.9 or one having a magnification of 50× and a numerical aperture of 0.5 was used. A power of the laser light with which the sample surface is irradiated through the objective lens 13 was 60 μW.

Raman scattered light (SERS light) Ls generated in response to the irradiation of the excitation light LP and collected by the objective lens 13 was transmitted through the dichroic mirror 12 and an optical filter 14 and was incident on a spectroscope 15. The spectroscope 15 includes a cooled CCD detector, and a spectrum of the SERS light was measured by the spectroscope 15.

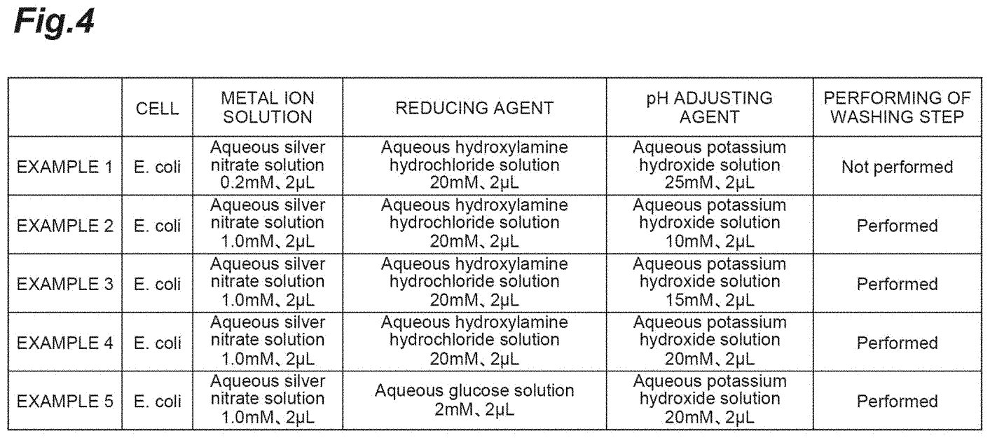

FIG. 4 is a table showing samples used in the respective examples. In each example, E coli (DH5α competent cell) was used as the cell being the analyte, and the cells were dispersed in ultrapure water to prepare the measurement solution.

In the example 1, an aqueous silver nitrate solution (concentration 0.2 mM) was used as the metal ion solution, an aqueous hydroxylamine hydrochloride solution (concentration 20 mM) was used as the reducing agent, and an aqueous potassium hydroxide solution (concentration 25 mM) was used as the pH adjusting agent. The procedure in the example 1 was based on the cell analysis method of the first embodiment (FIG. 1), as described below.

In the mixing step S11, the measurement solution, the metal ion solution, and the pH adjusting agent were adjusted to respective predetermined concentrations. On the glass slide serving as the support, 2 μL of the metal ion solution was dropped, and 2 μL of the measurement solution was further dropped onto the dropped spot, and these solutions were mixed on the glass slide. 2 μL of the reducing agent was further dropped onto the dropped spot, and these were mixed on the glass slide. Then, 2 μL of the pH adjusting agent was further dropped onto the dropped spot, and these were mixed on the glass slide to prepare the mixture solution.

In the metal microstructure generation step S12, the liquid droplet on the glass slide was allowed to stand still for an hour in a humidified environment, the metal ions were reduced by the reducing action of the reducing agent in the mixture solution to generate the metal microstructure on the glass slide, and the cell or the cell-derived substance was attached to the metal microstructure. After standing still for an hour in the metal microstructure generation step S12, the glass slide was dried in the drying step S13.

In the measurement step S15, the metal microstructure on the glass slide was irradiated with the excitation light, and the spectrum of the Raman scattered light (SERS light) generated by the excitation light irradiation was measured. In this step, using the microspectroscope, the metal microstructure was irradiated with the excitation light through the objective lens, and the spectrum of the SERS light was measured through the objective lens.

In the examples 2 to 4, the measurement condition is different from that in the example 1 in the concentrations of the metal ion solution and the pH adjusting agent. The concentration of the metal ion solution (aqueous silver nitrate solution) in the examples 2 to 4 was 1.0 mM. The concentration of the reducing agent (aqueous hydroxylamine hydrochloride solution) in the examples 2 to 4 was 20 mM as in the example 1. The concentration of the pH adjusting agent (aqueous potassium hydroxide solution) in the example 2 was 10 mM, the concentration of the pH adjusting agent in the example 3 was 15 mM, and the concentration of the pH adjusting agent in the example 4 was 20 mM.

Further, in the examples 2 to 4, the measurement condition is different from that in the example 1 in that the procedure of the cell analysis method of the second embodiment (FIG. 2) is used (that is, the washing step S14 is performed). The procedures of the mixing step

S11, the metal microstructure generation step S12, the drying step S13, and the measurement step S15 in the examples 2 to 4 were the same as those in the example 1.

In the example 5, the measurement condition is different from that in the example 4 in that an aqueous glucose solution (concentration 2 mM) was used as the reducing agent. An aqueous silver nitrate solution (concentration 1.0 mM) was used as the metal ion solution, an aqueous glucose solution (concentration 2 mM) was used as the reducing agent, and an aqueous potassium hydroxide solution (concentration 20 mM) was used as the pH adjusting agent. The procedure in the example 5 was same as that in the examples 2 to 4.

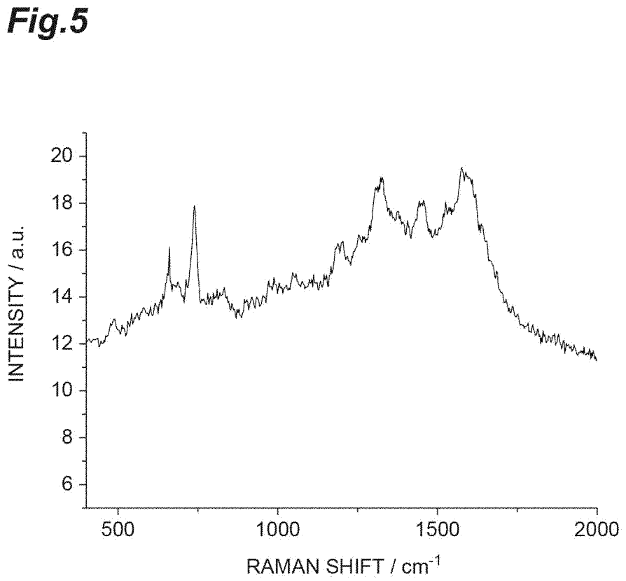

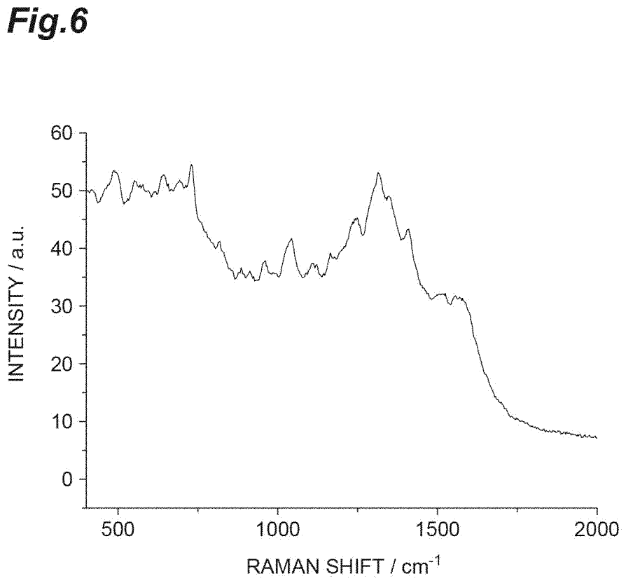

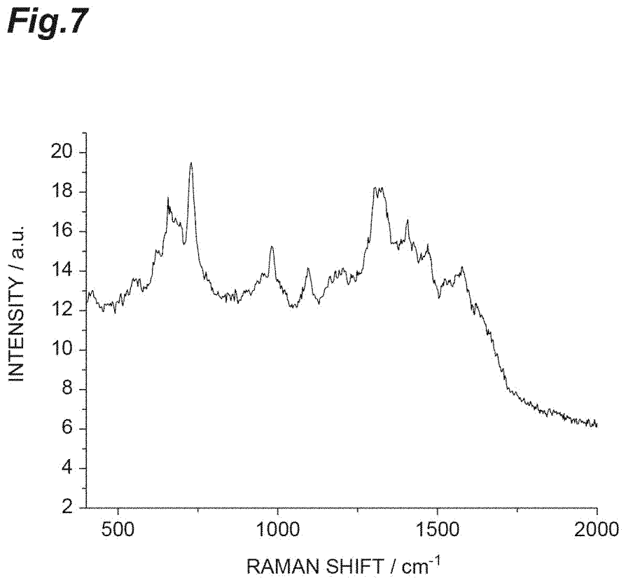

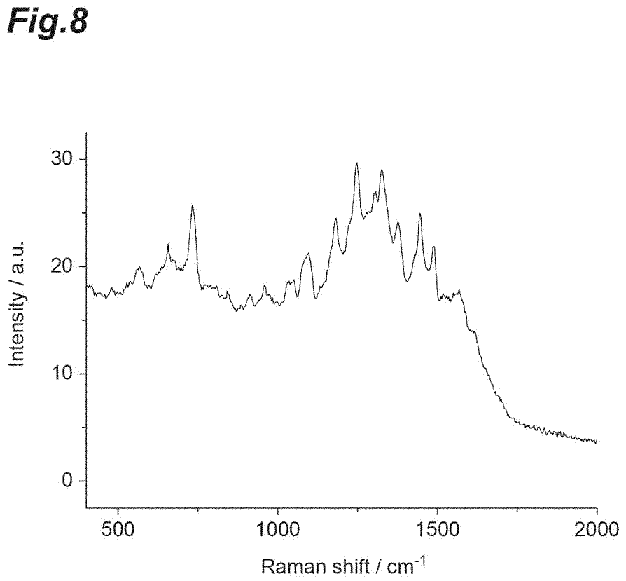

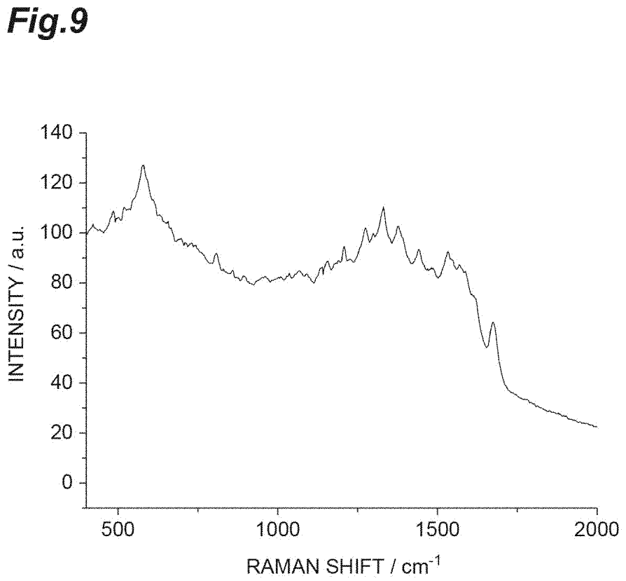

FIG. 5 is a diagram showing the SERS light spectrum obtained in the example 1. FIG. 6 is a diagram showing the SERS light spectrum obtained in the example 2. FIG. 7 is a diagram showing the SERS light spectrum obtained in the example 3. FIG. 8 is a diagram showing the SERS light spectrum obtained in the example 4. FIG. 9 is a diagram showing the SERS light spectrum obtained in the example 5. In these diagrams, the horizontal axis represents a Raman shift amount (unit cm⁻¹), and the vertical axis represents a Raman scattering intensity (arb. unit).

According to the description in Non Patent Document 1, the

SERS light spectrum of the cell-derived substance can also be obtained by using metal colloid particles. The measured SERS light is generated by contents such as nucleic acid and nucleic acid base contained in the cell or metabolites thereof, and the acquired SERS light spectrum is considered to have information of these.

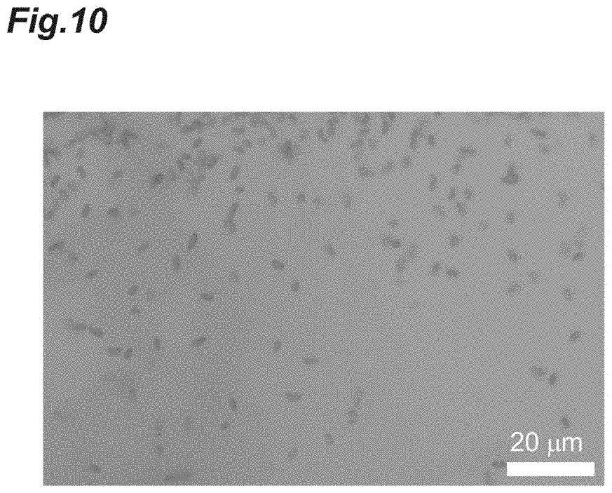

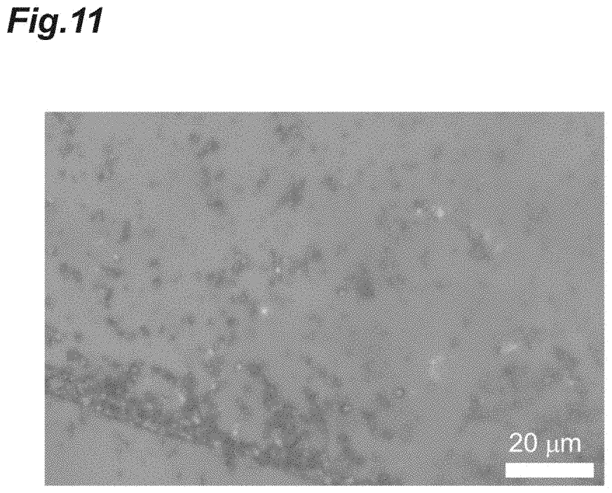

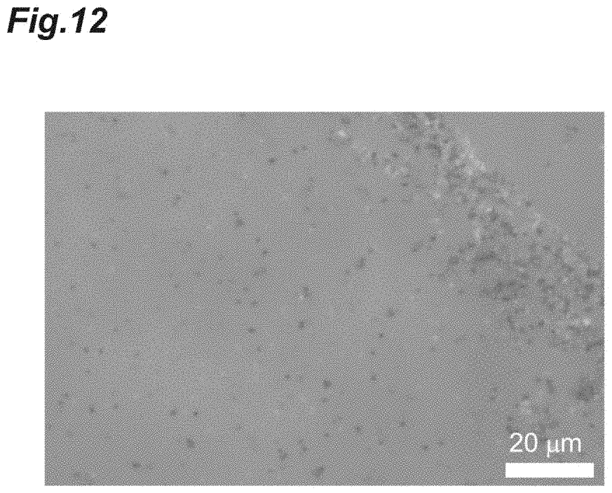

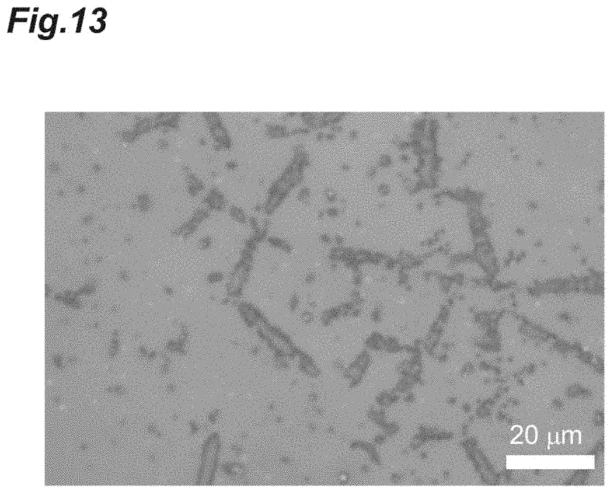

FIG. 10 is a photograph of a bright field image in a comparative example. In the comparative example, the glass slide onto which the measurement solution was dropped was dried without generating the metal microstructure, the glass slide was washed, and the sample after the washing was imaged. FIG. 11 is a photograph of a bright field image in the measurement step in the example 2. FIG. 12 is a photograph of a bright field image in the measurement step in the example 3. FIG. 13 is a photograph of a bright field image in the measurement step in the example 4.

In the image of the comparative example (FIG. 10), the shape of the cell attached to the glass slide can be identified. On the other hand, in the images of the examples (FIG. 11 to FIG. 13), the shape of the cell being the analyte cannot be identified, and it is considered that the cell is degraded. Further, in the images of the examples (FIG. 11 to FIG. 13), bright spots due to a part of the degraded cells and silver microparticles are observed.

Each of the SERS light spectra in the examples 2 to 5 (FIG. 6 to FIG. 9) has a large number of peaks. This is considered to be due to the fact that, in the examples 2 to 5, the mixture solution was made alkaline by the pH adjusting agent, so that lysis of the cell was promoted as shown in the bright field image photographs (FIG. 11 to FIG. 13), and many contents thereof were observed.

As described above, in the cell analysis method of the present embodiment, the metal ions in the mixture solution are reduced by the reducing action of the reducing agent in the mixture solution to generate the metal microstructure on the support, the cell or the cell-derived substance is attached to the metal microstructure, the spectrum of the Raman scattered light (SERS light) generated by the excitation light irradiation thereon is measured, and the cell is analyzed based on the spectrum. As compared with the conventional analysis method, the cell analysis method of the present embodiment can perform the analysis simply and quickly.

In the conventional analysis method, the analytes that can be subjected to the SERS spectroscopy are limited to those that have high affinity for the metal constituting the metal microstructure and are easily adsorbed. Further, in the invention disclosed in Patent Document 1, the analytes that can be subjected to the SERS spectroscopy are limited to those having reducing action. In contrast, in the cell analysis method of the present embodiment, it is possible to form the metal microstructure even with the cell that has low affinity for the metal constituting the metal microstructure and that is difficult to be adsorbed or with the cell that does not have reducing action, and the cell or the cell-derived substance can enter a narrow gap in the metal microstructure, and thus the second condition can be satisfied, and this makes it possible to analyze the cell by the SERS spectroscopy.

In the conventional analysis method, it is necessary to prepare a SERS substrate or metal colloids in advance for performing SERS light spectrum measurement. In contrast, in the cell analysis method of the present embodiment, it is possible to generate the metal microstructure and to attach the cell (or the cell-derived substance) to the metal microstructure immediately before SERS light spectrum measurement. Therefore, in the cell analysis method of the present embodiment, even in a case where silver, which is easily oxidized, is used to generate the metal microstructure, it is possible to suppress oxidization of silver, and to perform efficient SERS spectroscopy.

In the cell analysis method of the present embodiment, it is not necessary to prepare the SERS substrate or metal colloids in advance, and therefore, it is free from the problem of contamination of these, thereby making it possible to easily analyze the cell. Further, the cell analysis method of the present embodiment uses the metal ion solution, which is available at a lower cost than the SERS substrate and metal colloids, and also for this reason, it is possible to easily perform the analysis of the cell.

In the analysis method using a metal colloid dispersion liquid described in Non Patent Document 1, the SERS spectroscopy is difficult when an amount of cells is very small. In contrast, in the cell analysis method of the present embodiment, the SERS spectroscopy can be performed even when an amount of cells is very small.

Further, in the analysis method described in Non Patent Document 1, S light spectrum measurement is performed by covering a cell with metal colloids, and it is necessary to find out the cell with a microscope at the time of the measurement, and thus, the measurement is not easy. In contrast, in the cell analysis method of the present embodiment (in particular, the second embodiment), since the SERS light spectrum measurement is performed by lysing, and further drying and washing the cell, and adsorbing the cell-derived contents to the metal microstructure, the measurement is easy.

The cell analysis method is not limited to the embodiments and configuration examples described above, and can be modified in various ways.

The cell analysis method of the above embodiment includes (1) a mixing step of mixing a cell as an analyte, a metal ion solution, and a reducing agent to prepare a mixture solution; (2) a metal microstructure generation step of reducing metal ions in the mixture solution by reducing action of the reducing agent in the mixture solution to generate a metal microstructure on a support, and attaching the cell or a cell-derived substance to the metal microstructure; (3) a drying step of, after the metal microstructure generation step, drying the support; and (4) a measurement step of, after the drying step, irradiating the metal microstructure on the support with excitation light, and measuring a spectrum of Raman scattered light generated by the excitation light irradiation.

The above cell analysis method may further include, between the drying step and the measurement step, a washing step of washing the support. In this case, the cell analysis method includes (1) a mixing step of mixing a cell as an analyte, a metal ion solution, and a reducing agent to prepare a mixture solution; (2) a metal microstructure generation step of reducing metal ions in the mixture solution by reducing action of the reducing agent in the mixture solution to generate a metal microstructure on a support, and attaching the cell or a cell-derived substance to the metal microstructure; (3) a drying step of, after the metal microstructure generation step, drying the support; (4) a washing step of, after the drying step, washing the support; and (5) a measurement step of, after the washing step, irradiating the metal microstructure on the support with excitation light, and measuring a spectrum of Raman scattered light generated by the excitation light irradiation.

In the above cell analysis method, in the mixing step, a pH adjusting agent may be further mixed to prepare the mixture solution.

### INDUSTRIAL APPLICABILITY

The present invention can be used as a method capable of easily performing an analysis on a cell being an analyte by highly efficient SERS spectroscopy.

### REFERENCE SIGNS LIST

1—microspectroscope, 11—excitation light source, 12—dichroic mirror, 13—objective lens, 14—optical filter, 15—spectroscope, 21—support, 22—metal microstructure, 23—cell (or cell-derived substance).

1. A cell analysis method comprising:
a mixing step of mixing a cell as an analyte, a metal ion solution, and a reducing agent to prepare a mixture solution;
a metal microstructure generation step of reducing metal ions in the mixture solution by reducing action of the reducing agent in the mixture solution to generate a metal microstructure on a support, and attaching the cell or a cell-derived substance to the metal microstructure;
a drying step of, after the metal microstructure generation step, drying the support; and
a measurement step of, after the drying step, irradiating the metal microstructure on the support with excitation light, and measuring a spectrum of Raman scattered light generated by the excitation light irradiation.

2. The cell analysis method according to claim 1, further comprising, between the drying step and the measurement step, a washing step of washing the support.

3. The cell analysis method according to claim 1, wherein, in the mixing step, a pH adjusting agent is further mixed to prepare the mixture solution.
